(19) United States
(12) Patent Application Publication
(10) Pub. No.: US 2004/0236298 A1
(43) Pub. Date: Nov. 25, 2004

(54) Disposable and reusable protective undergarments

(76) Inventor: Fredrica V Coates, Winston-Salem, NC (US)

Correspondence Address: RATNERPRESTIA, P O BOX 980, VALLEY FORGE, PA, 19482-0980, US

(21) Appl. No.: 10/483,523

(22) PCT Filed: Jul. 15, 2002
(86) PCT No.: PCT/US02/22530

(30) Foreign Application Priority Data
Jul. 13, 2001 (US) 60304790
Jul. 13, 2001 (US) 60304792
Jul. 13, 2001 (US) 60304789
Jul. 13, 2001 (US) 60304769
Jul. 13, 2001 (US) 60304788
Jul. 13, 2001 (US) 60304787
Jul. 13, 2001 (US) 60304791
Jul. 13, 2001 (US) 60304770

(51) Int. Cl. A61F 13/15; A61F 13/20

(52) U.S. Cl. 604/385.04

(57) ABSTRACT

An absorbent article is provided according to exemplary aspects of this invention. The absorbent article includes a body portion having a perimeter and a garment-facing surface. The absorbent article also includes a pair of fastening tabs extending from the garment-facing surface of the body portion at locations spaced inwardly from the perimeter of the body portion. Each of the fastening tabs has means for engaging the other of the fastening tabs, and the fastening tabs are positioned to receive a portion of a garment therebetween.

### FIELD OF THE INVENTION

This invention relates to absorbent articles and, more particularly, to disposable and reusable protective undergarments.

### BACKGROUND OF THE INVENTION

The use of absorbent articles, such as protective undergarments, has increased steadily for many years. Early uses, such as disposable diapers for babies and disposable sanitary products for women, have in recent years been joined by an increasing demand for products designed to deal with incontinence issues for adults, frequently brought on by advanced age, obesity, or a variety of medical conditions. At the same time, markets have grown in all of these areas for both disposable and reusable products, depending on the preferences of the consumer.

Disposable absorbent articles, such as diapers and pads for example, are in widespread use throughout the world as a result of their convenience. They provide substantial advantages and convenience over absorbent articles that have to be laundered and reused, particularly when the absorbent articles are used away from home. In recent years, many different disposable absorbent articles have been proposed and some have been very successful in the marketplace. However, even current successful products leave room for improvement.

To promote preservation of the ecology, some consumers desire to return to the use of reusable, rather than disposable, absorbent articles such as infant and adult diapers. A recent improvement to reusable diapers is in the replacement of pin fasteners by fasteners of filamentary material, such as hook and loop filamentary materials manufactured by Velcro Corporation and Aplix Corporation. In this regard, reference is made to U.S. Pat. No. 4,537,591 to Coates, incorporated herein by reference, which discloses a cloth diaper having filamentary fasteners together with a self-closing tab cover that protects the fasteners from buildup of lint during washing.

Whether for reusable or disposable products, various fastening systems have been employed for fastening the absorbent products to the wearer or to the clothing of the wearer. For example, the waistband of a diaper is preferably fastened around the waist of the wearer, and the fastening system is generally intended to hold the diaper in snug encircling fashion on the wearer's torso. After the diaper is soiled, it is removed by unfastening the tabs, thereby opening the waist.

Although improvements have been made over the years relative to fastening systems for reusable and disposable absorbent articles, there remains a constant effort to reduce skin irritation for the user. In addition, systems continue to be sought for use by individuals with limited dexterity, perhaps due to a debilitating medical condition.

Similar needs arise in connection with feminine hygiene products as with infant or adult incontinence products. Sanitary napkins and pads, for example, such as absorbent devices designed to receive and contain vaginal discharges such as menses, are omnipresent items of modern society. Disposable sanitary napkins and pads designed to be held adjacent to the human body through the agency of a garment, such as an undergarment or panty, have effectively supplanted prior products that required a specially designed belt for use.

Panty pads, whether used for absorbing urine or blood, can lead to accidents and potential embarrassment for the wearer should they slip out of position during use. This slipping or other movement of the pad usually is due to an excessive amount of movement in that area of the body where the legs attach to the pubis area. Hence the pull or motion of the legs can dislodge the pad. Adding to these difficulties is the fact that women's styles change rapidly, an example being a recent trend toward the use of so-called “thong” panties, which have a narrow crotch design that is incompatible with the arrangement of most traditional feminine pads.

It will be appreciated from the above discussion that improvements in the design of fastening systems are needed for a variety of absorbent articles including protective undergarments such as diapers, incontinence products, and feminine hygiene pads.

### SUMMARY OF THE INVENTION

An absorbent article is provided according to exemplary aspects of this invention. The absorbent article includes a body portion having a perimeter and a garment-facing surface. The absorbent article also includes a pair of fastening tabs extending from the garment-facing surface of the body portion at locations spaced inwardly from the perimeter of the body portion. Each of the fastening tabs has means for engaging the other of the fastening tabs, and the fastening tabs are positioned to receive a portion of a garment between them.

A fastening tab for an absorbent article is also provided according to an exemplary aspect of this invention. The fastening tab includes opposite surfaces. One of the surfaces carries hooks and the other of the surfaces is engageable by hooks. The hooks are spaced from at least one edge portion of the fastening tab.

### BRIEF DESCRIPTION OF THE DRAWINGS

The invention is best understood from the following detailed description when read in connection with the accompanying drawings. It is emphasized that, according to common practice, the various features of the drawings are not rendered to any particular proportion or scale. On the contrary, the dimensions of the various features are arbitrarily expanded or reduced for clarity. Included in the drawings are the following figures:

FIG. 1 is a plan view of one side of an exemplary embodiment of an absorbent pad according to an aspect of the invention. FIG. 1A shows additional detail.

FIG. 2 is a plan view of one side of another exemplary embodiment of an absorbent pad. FIG. 2A shows additional detail.

FIG. 3 is a plan view of the opposite side of the absorbent pad shown in FIG. 2. FIG. 3A shows additional detail.

FIG. 4 is a plan view of one side of yet another exemplary embodiment of an absorbent pad according to an aspect of the invention.

FIG. 5 is a plan view of the opposite side of the absorbent pad shown in FIG. 4. FIG. 5A shows additional detail.

FIG. 6 is a perspective view of the absorbent pad shown in FIG. 4. FIG. 6A shows additional detail.

FIG. 7 is a plan view of one side of still another exemplary embodiment of an absorbent pad according to an aspect of the invention.

FIG. 8 is a plan view of one side of another exemplary embodiment of an absorbent pad according to an aspect of the invention. FIGS. 8A and 8B show additional detail.

FIG. 9 is a plan view of one side of yet another exemplary embodiment of an absorbent pad according to an aspect of the invention.

FIG. 10 is a perspective view of the absorbent pad shown in FIG. 9. FIG. 10A shows additional detail.

FIG. 11 is a perspective view of another exemplary embodiment of an absorbent pad according to an aspect of the invention. FIG. 11A shows additional detail.

FIG. 12 is another perspective view of the absorbent pad of FIG. 11, showing fastening tabs attached to one another. FIG. 12A shows additional detail.

FIG. 13 is a plan view of an exemplary embodiment of an absorbent pad according to an aspect of the invention, showing a device capable of collapsing to a folded position for storage.

FIG. 14 is a plan view of the absorbent pad of FIG. 13, showing fastening tabs attached to each other as they would be in attaching the device to an undergarment (not shown).

FIG. 15 is a plan view of the absorbent pad of FIG. 13 in a folded position. FIGS. 15A-15G shows additional detail.

FIG. 16 is a plan view of another exemplary embodiment of an absorbent article according to an aspect of the invention, showing broad lateral fastening tabs, one of which is folded over to illustrate features on the opposite side of that tab. FIG. 16A shows additional detail.

FIG. 17 is a plan view of another exemplary embodiment of an absorbent pad according to an aspect of the invention, showing a pad configured to allow folding for storage. FIG. 17A shows additional detail.

FIG. 18 is a plan view of the absorbent pad of FIG. 17, showing the device folded in storage configuration. FIG. 18A shows additional detail.

FIG. 19 is an exploded, perspective view of an exemplary embodiment of an absorbent article according to another aspect of the invention, showing a pad and pouch system with the two elements separated from one another. FIG. 19A shows additional detail.

FIG. 20 is a perspective view of the article of FIG. 19, showing the pad and pouch elements together. FIG. 20A shows additional detail.

FIG. 21 is a plan view of another exemplary embodiment of an absorbent pad according to an aspect of the invention, showing a pad with pairs of fastening tabs. FIG. 21A shows additional detail.

FIG. 22 is a plan view of the absorbent pad of FIG. 21, showing the fastening tabs attached to one another. FIG. 22A shows additional detail.

FIG. 23 is a plan view of yet another exemplary embodiment of an absorbent pad according to an aspect of the invention, with one fastening tab extended and the other folded to reveal features provided on opposed sides of one or both of the tabs. FIG. 23A shows additional detail.

FIG. 24 is a plan view of another exemplary embodiment of an absorbent pad according to an aspect of the invention, similar to that shown in FIG. 21 having pairs of fastening tabs.

FIG. 25 is a plan view of an opposite side of the pad of FIG. 24 showing the fastening tabs attached to one another.

### DETAILED DESCRIPTION OF THE INVENTION

Exemplary features of selected embodiments of this invention will now be described with reference to the figures. It will be appreciated that the spirit and scope of the invention is not limited to the embodiments selected for illustration. Also, it should be noted that the drawings are not rendered to any particular scale or proportion. It is contemplated that any of the configurations and materials described hereafter can be modified within the scope of this invention.

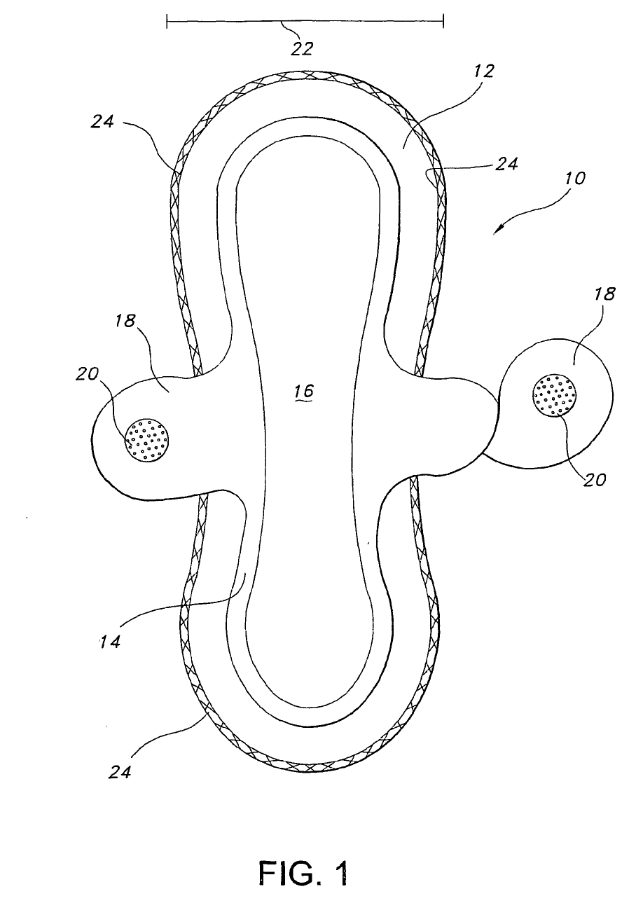

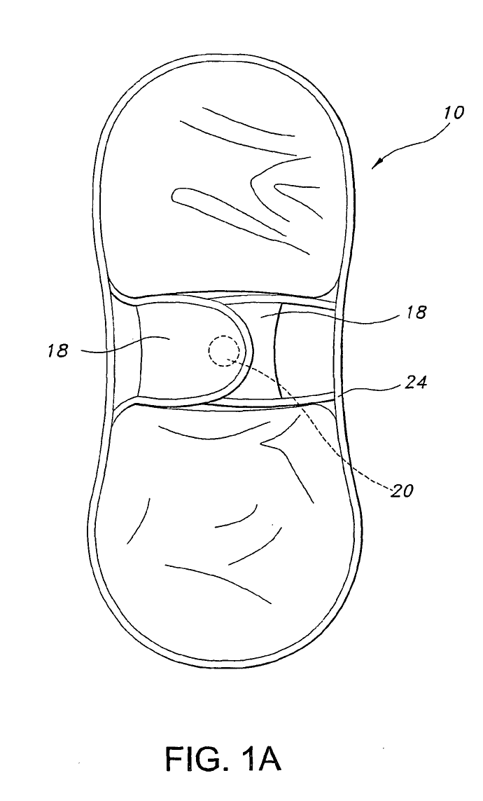

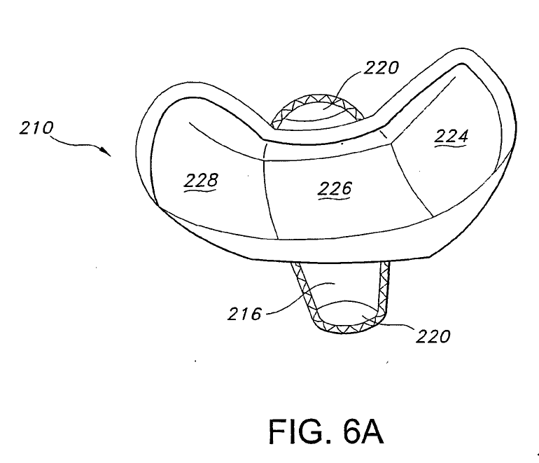

Referring to FIGS. 1 through 25, an absorbent article is provided with an improved fastening system. Additional related figures are provided to show additional detail. Specifically, FIGS. 1A, 2A, 3A, 5A, 6A, 8A and 8B, 10A, 11A, 12A, 15A-15G, 16A, 17A, 18A, 19A, 20A, 21A, 22A, and 23A show additional detail corresponding to FIGS. 1, 2, 3, 5, 6, 8, 10, 11, 12, 15, 16, 17, 18, 19, 20, 21, 22, and 23, respectively.

Generally, the absorbent article (such as article 10 illustrated in FIG. 1 and FIG. 1A) includes a body portion having a perimeter and a garment-facing surface. The absorbent article also includes a pair of fastening tabs extending from the garment-facing surface of the body portion at locations spaced inwardly from the perimeter of the body portion. Each of the fastening tabs has means for engaging the other of the fastening tabs, and the fastening tabs are positioned to receive a portion of a garment between them.

The engaging means optionally includes an adhesive, hooks, or a snap. For example, the fastening tabs can each include a surface carrying hooks and a surface engageable by hooks. Such surfaces include, but are not limited to, woven and non-woven materials comprising polypropylene, polyethylene, polyester, NYLON, or RAYON. Advantageously, the hooks may be engageable by loops. Such products are for example by the Velcro Corporation under the mark VELCRO. The fastening tabs are optionally end portions of a single component of the absorbent article. Alternatively, the fastening tabs are separate from one another. The fastening tabs can be coupled to, or integral with, a layer of the body portion.

The body portion has a width, and the space between the fastening tabs can be selected to be smaller than the width of the body portion. For example, the space between the fastening tabs can be selected to receive the crotch portion of a thong-style garment.

According to another aspect of the invention, a fastening tab is provided for an absorbent article. The fastening tab includes opposite surfaces, one of which carries hooks and the other of which is engageable by hooks. The hooks are spaced from at least one edge portion of the fastening tab.

The fastening tab optionally includes an elastic material. Additionally, the fastening tab can optionally include a woven or non-woven substrate layer from which hooks extend, wherein the substrate is engageable by hooks. If used, the hooks are optionally positioned in a region to define a border between an edge portion of the fastening tab and the hooks, wherein the border of the fastening tab is devoid of hooks and the border substantially surrounds the region in which the hooks are positioned. The region of the hooks can be substantially circular and/or positioned at an end portion of the fastening tab. The edge portion of the fastening tabs can be provided with an arcuate shape for comfort.

Depending on cost, performance requirements, particular uses, and manufacturing considerations, various materials can be selected for use in absorbent articles according to this invention. U.S. Pat. No. 6,004,893 to Van Tilburg, which is incorporated herein by reference, describes a variety of such materials and associated constructions.

For example, the absorbent article can be provided with a topsheet that is liquid permeable and, when the article is in use, is in close proximity to the skin of the user. If used, the topsheet is preferably compliant, soft feeling and non-irritating to the user's skin. Such topsheets can be made from any of the materials conventional for this type of use. Non-limiting examples of suitable materials that can be used as a topsheet are woven and non-woven polyester, polypropylene, polyethylene, NYLON, and RAYON and formed thermoplastic films. Suitable films are described, for example, in U.S. Pat. No. 4,324,246 to Mullane and Smith and U.S. Pat. No. 4,342,314 to Radel and Thompson, both of which patents are incorporated herein by reference. Formed films may be selected for the topsheet because they are pervious to liquids and yet non-absorbent. Thus, the surface of the formed film, which is in contact with the body, remains substantially dry and is more comfortable to the wearer.

In exemplary embodiments, the inner surface of the topsheet can be secured in contacting relation to an absorbent core. This contacting relationship results in liquid penetrating the topsheet faster than if it were not in contact with the absorbent core. The topsheet can be maintained in contact with the absorbent core by applying adhesive, optionally in spaced, limited areas, to an inner surface of the topsheet. Examples of suitable adhesives used for this purpose include the acrylic emulsion E-1833BT manufactured by Rohm and Haas Company of Philadelphia, Pa. and acrylic emulsions manufactured by H. B. Fuller Company of St. Paul, Minn. Also contemplated are thermoplastic hot melt adhesives such as 34-563A, available from National Starch, Inc.

The absorbent core is generally compressible, conformable, and non-irritating to the user's skin. It can comprise any material used in the art for such purpose. Examples of suitable materials are layers of tissue (such as wadding) and fibrated comminution pulp (e.g., airfelt). Also contemplated are cotton, cellulose acetate, and any of these in combination with a superabsorbent polymer.

The absorbent article preferably includes a backsheet that is preferably impervious to liquids and, thus, prevents menstrual fluid that may be expressed from the absorbent core from soiling the clothing of the user. Any material used in the art for such purposes can be utilized herein. Suitable materials include embossed or non-embossed polyethylene and polypropylene films and laminated tissue and non-woven materials.

One or more-outer surfaces of the backsheet can be coated with an adhesive for securing the absorbent article in the crotch portion of a panty. Any adhesive or glue used in the art for such purpose can be used. For example, pressure sensitive adhesives are optionally selected. Suitable adhesives include INSTANT LOK adhesive products manufactured by National Starch Company. The pressure sensitive adhesive of the central pad adhesive is preferably covered with a release liner to keep the adhesive from drying out and to keep it from sticking to extraneous surfaces prior to use. Any commercially available release liner commonly used for such purposes can be used herein. A non-limiting example of suitable release liners is BL 30 MG-A Silox, which is manufactured by the Akrosil division of International Paper, Inc.

Exemplary materials and constructions for reusable absorbent articles are described in U.S. Pat. No. 5,891,122 and U.S. Pat. No. 6,254,583, both issued to Coates, and both of which are incorporated herein by reference. Additional materials and constructions are well known in the art of reusable absorbent articles.

Referring now to FIGS. 1 through 25, features of exemplary embodiments of absorbent articles have been selected for illustration and description herein. Although selected embodiments are illustrated, other embodiments are contemplated within the spirit and scope of this invention. Also, although each of the selected embodiments is configured for use as an absorbent pad in hygiene and incontinence applications, this invention applies with equal benefit to other absorbent articles including diapers, incontinence garments, and other products utilized to absorb bodily and other fluids. Such articles may include for example adult and baby diapers, adult and children's absorbent underwear, panty liners, panty shields, maxi pads, mini pads, breast pads, wound dressings, and baby bibs.

Referring to FIG. 1, an embodiment is disclosed in the form of a three-layer disposable panty liner for fashion-type undergarments. In this embodiment, hook and loop type fasteners are used to attach the liner to the panties. The first, inner layer is absorbent, the second is both waterproof and is attached to the attachment loop portions, and the third, outermost layer is another waterproof layer. In addition, it may be advantageous to add a body-facing layer of a material designed to keep moisture in the absorbent layer out of direct contact with the skin. The garment has elasticized sides and the layers are of narrow width so that they may be attached to the fashion-type undergarment in the usual way. The embodiment illustrated in FIG. 1 is configured for use during a woman's menstrual period and is especially designed to be attached to undergarments of the fashionable type, which are thinner in the crotch area than regular panties.

Details of the embodiment illustrated in FIG. 1 are provided in U.S. Provisional Patent Application Serial No. 60/304,790, filed Jul. 13, 2001 and entitled DISPOSABLE PANTY-LINER, which is incorporated herein by reference. FIG. 1 shows the three layers and the die-cut attachment loops with double-sided hook and loop type attachment means. The exemplary absorbent article, in the form of a disposable panty liner, is generally indicated at 10 in FIG. 1. There is an outer, waterproof layer 12, a second to waterproof layer 14, and the absorbent inner layer 16. In addition, it may be advantageous to add a body-facing layer of a material designed to keep moisture in the absorbent layer out of direct contact with the skin. On the second layer 14 are attached the die-cut fastening tabs, indicated at 18. Located on these tabs are double-sided hook and loop-type fasteners 20 that are used to attach the device to an undergarment.

It should be noted that in FIG. 1, one tab is partially turned over so that the other side of the tab with the attendant fastener 20 is exposed. Double-sided fasteners of this type are configured to engage with the opposite, interengaging fastener type (hook with loop and vice versa). The tabs 18 are wrapped around the crotch of an undergarment (not shown) and are engaged with one another. Note that the fasteners 20 in this exemplary embodiment are substantially circular, relatively small, and optionally coin shaped. Other shapes may also be used.

As the type of undergarment envisaged for use with the embodiment shown in FIG. 1 is a “fashion type” or “thong” undergarment having a narrower crotch area than “regular” undergarments, the various layers 12, 14, and 16 are relatively narrow in width, as is indicated by line 22. The outer portions of the liner 10 are elasticized at 24 to allow for a comfortable, body hugging fit with good protection along with providing a full pad in the primary or inner layer 16.

The embodiment illustrated in FIG. 1 features attachment of at least one fastening tab to a third layer of narrow width, a width optionally as narrow as the crotch of fashion underwear. It is recognized that existing panty liners and pads do not always work well with thong-type and narrower crotch underwear. The embodiment illustrated in FIG. 1 can be fastened to such panties while still allowing full-pad protection by the primary layer. This is accomplished because the absorbent article embodiment illustrated in FIG. 1 includes a pair of fastening tabs that extend from a garment-facing surface of the article's body portion at locations that are spaced inwardly from the perimeter of the body portion. The secondary layer may be optionally utilized for fastening only and may or may not be waterproof. The sides are optionally elasticized (as illustrated) to create a body-hugging curve to provide a more comfortable product with greater protection.

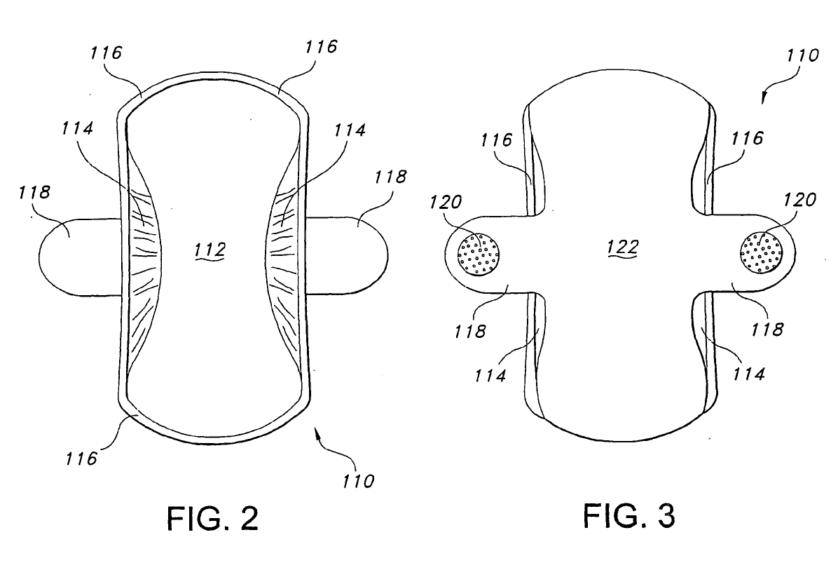

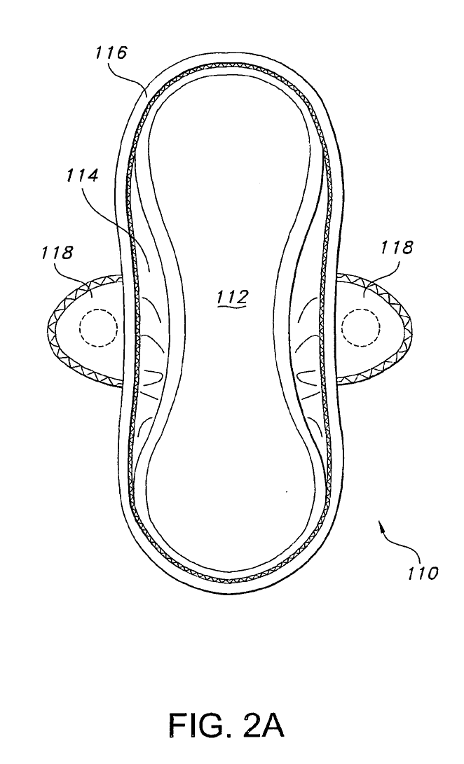

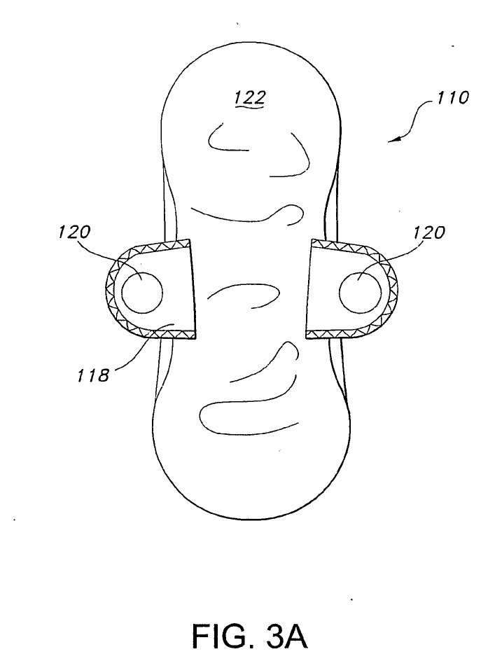

Referring now to FIGS. 2 and 3, an embodiment is illustrated in the form of a triple layer panty-liner having double-sided hook and loop fasteners located on laterally disposed fastening tabs. The liner includes a decorative outer edge and the device may be adapted to be reusable, i.e., it may comprise an undergarment or pad that is washable for repeated use. The illustrated hook and loop fastener arrangement allows the user to lay either the right or the left fastening tab over the other, whichever is more convenient. The illustrated embodiment is configured for use as a menstrual or incontinence pad or liner.

Details of the embodiment illustrated in FIGS. 2 and 3 are provided in U.S. Provisional Patent Application Serial No. 60/305,879, filed Jul. 18, 2001 and entitled FASHION PANTY-LINER, which is incorporated herein by reference. Referring to FIG. 2, this embodiment of an absorbent article, in the form of a panty-liner, is indicated generally at 110. The top side of the liner is seen in FIG. 2 showing the absorbent portion 112 surrounded by a gusset 114. The gusset 114 is made from a waterproof material that channels fluid toward the absorbent portion 112. Located peripherally around the gusset 114 is a decorative designer elastic 116.

A pair of fastening tabs 118 is laterally disposed on the liner as is best seen in FIG. 3. Located on the tabs 118 are double-sided hook and loop fasteners 120. The fasteners 120 are attached to the waterproof layer 122 of the liner 110. The hook and loop fasteners 120 are configured so that either the left side tab can be placed over the right side tab or vice versa. This configuration makes attaching the device to an undergarment easier for the visually impaired or for a user suffering from arthritis or the like. The optional configuration of the liner 110 for reuse makes the product attractive to environmentally conscious customers. The embodiment illustrated in FIGS. 2 and 3 therefore exemplifies a pad product with wraparound fastening tabs that have a double-sided hook and loop closure system to hold the pad securely. Gussets provide protection from leakage at the legs. The pad optionally includes designer elastic around the full, outer perimeter edge of the pad. In addition, the positioning of absorbent portion 112 adjacent the wearer, opposite from the orientation shown in FIG. 1, may confer additional advantages in comfort and utility.

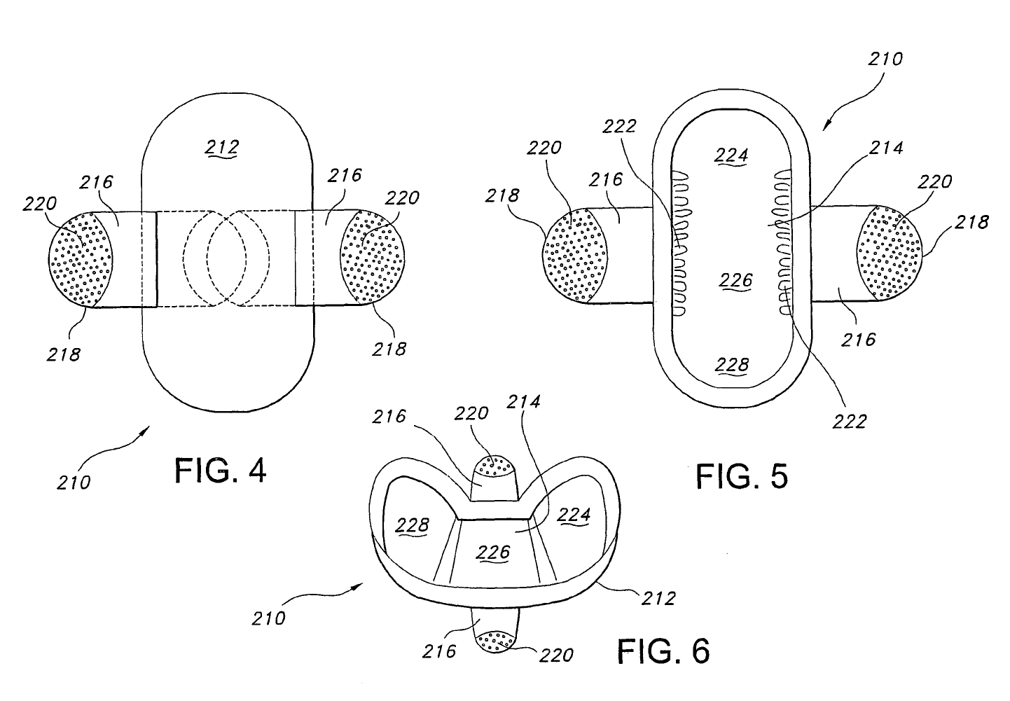

Another embodiment of an incontinence pad with wraparound double-sided hook and loop fasteners is disclosed in FIGS. 4-6. The illustrated device includes a waterproof backsheet, and the hook portion of the fastener is located on the outer perimeter of the wraparound fastening tabs. Additionally, the device also includes elasticized sides to provide a fit that conforms to the user's body.

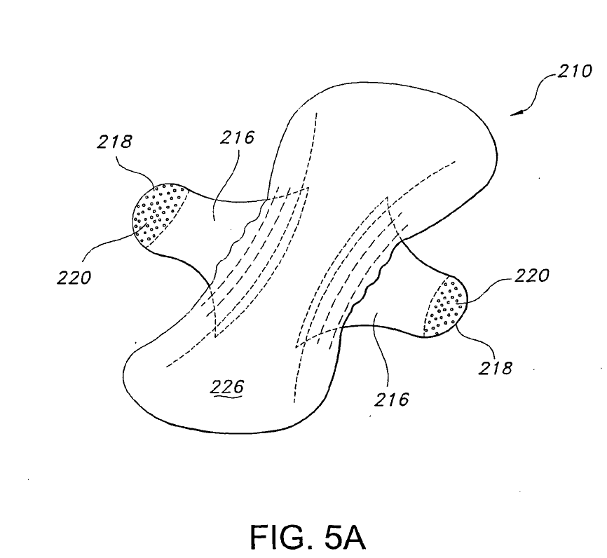

Details of the embodiment illustrated in FIGS. 4-6 are provided in U.S. Provisional Patent Application Serial No. 60/305,896, filed Jul. 18, 2001 and entitled PANTY-LINER, which is incorporated herein by reference. Referring to FIGS. 4-6, the disclosed embodiment of an incontinence pad is generally indicated at 210. The incontinence pad 210 has a bottom side 212, which is preferably waterproof, and a top side 214 (seen in FIG. 5) that is absorbent. The device has laterally disposed fastening tabs indicated at 216. Double-sided hook and loop fasteners are located on the tabs 216.

The material from which the tabs are made provides the loop material of the cooperating fasteners, and the hook portion of the fastener is attached on the outer perimeter edge 218 of the tabs 216, the hook portion of this embodiment being indicated at 220. The side walls of the pad 210 are elasticized, with the elasticized portion denoted at 222. The top side absorbent portion 214 has a front 224, a middle 226, and a back 228. The configuration of the double-sided hook and loop fasteners as described allows the user to fasten either the left side tab over the right tab or vice versa.

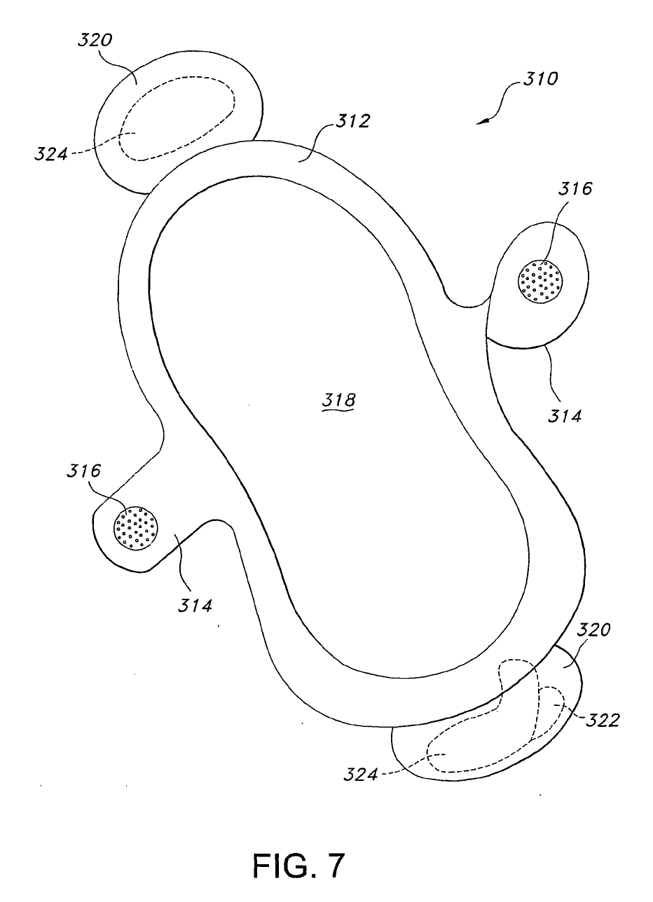

Referring now to FIG. 7, an embodiment of a panty liner is disclosed where side tabs having double-sided hook and loop type fasteners and front and rear peel-away adhesive tabs are used to secure the liner to an undergarment. Both hook and loop type fasteners are disposed on side tabs of the liner and peel-away adhesive tabs are located on the front and rear ends of the device to secure the liner to the undergarment of the user. Thus, when a woman desires or needs to use a longer pad on occasion, this longer pad can be firmly and safely secured in place. Although both front and rear adhesive tabs are shown in FIG. 7, the use of one or three or more tabs may be advantageous.

Details of the embodiment illustrated in FIG. 7 are provided in U.S. Provisional Patent Application Serial No. 60/304,792 filed Jul. 13, 2001 and entitled PANTY-LINER, which is incorporated herein by reference. FIG. 7 is a top view of the liner embodiment, showing the side tabs with their attendant double-sided hook and loop fasteners and also showing the front and rearwardly disposed adhesive portions with the peel-off areas covering the adhesive. The panty liner is generally indicated in FIG. 7 at 310. The liner has an outer portion or layer 312 that has disposed thereon the side tabs 314. Located on the side tabs 314 are the double-sided hook and loop type fasteners 316. The double-sided fasteners of this type have the hooks on one surface and the loops on the other such that the tabs 314 may be moved over the outer crotch area of the undergarment (not shown) and fastened together to secure the liner in place.

The fasteners 316 in the embodiment illustrated in FIG. 7 are small and substantially rounded or circular in shape. On the inner portion of the liner disclosed in FIG. 7 is the absorbent pad portion 318. The illustrated pad is a larger type that may be desirable during certain times of a woman's menstrual period. Because of the size of such pads, it is desirable to have additional fastening means to secure the device in place. This is optionally accomplished in the present invention by providing front and rear tabs 320 that include an adhesive portion 322 (seen in broken lines in FIG. 7) thereon to further secure the liner to an undergarment. These adhesive portions 322 are covered by a peel-away cover, also shown in broken lines, and indicated at 324. These adhesive portions are relatively small and are located far enough away from the main portion of liner 310 so as to preclude any accidental adhesion to body hair.

Another feature of the illustrated embodiment of FIG. 7 is that the double-sided fastener configuration disclosed allows the user to either put the right tab over the left or the left tab over the right, whichever is more convenient to the user. Accordingly, FIG. 7 illustrates an embodiment of a sanitary pad with tabs that wrap around the crotch of a panty and secure with a double-sided hook and loop fastener and have adhesive tabs on opposite frontal and rear ends to adhere to the panty.

Despite a trend toward the use of thinner pads, women still sometimes prefer to use longer pads on some days. Such pads may tend to roll up and expose the adhesive. The optional use of the small adhesive tabs of FIG. 7 at opposite ends, far away from the center of the pad, can help to prevent rolling and can help avoid accidental adhesion to body hair.

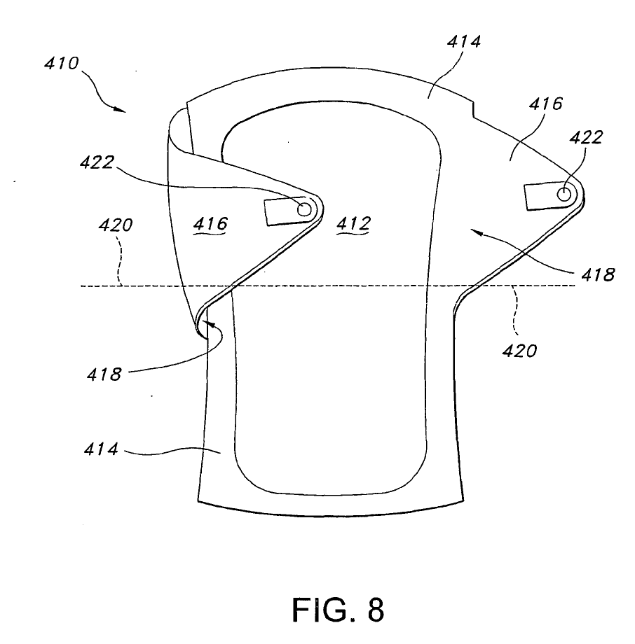

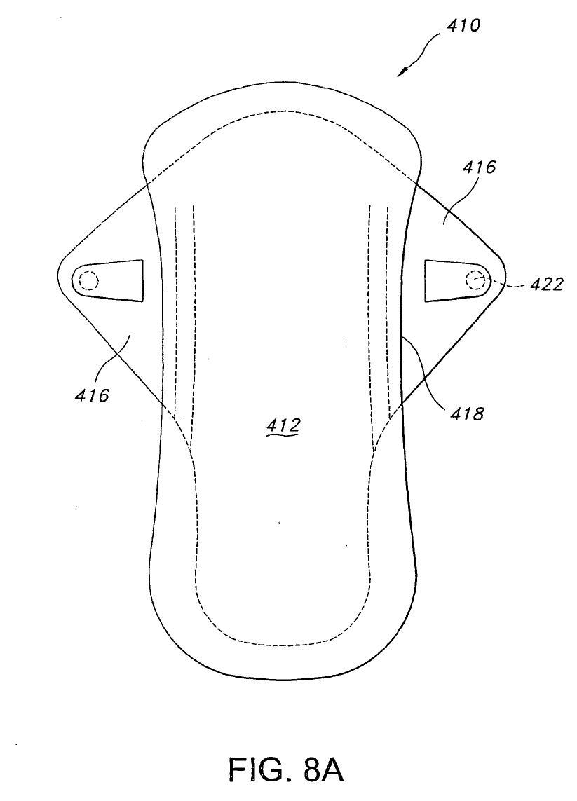

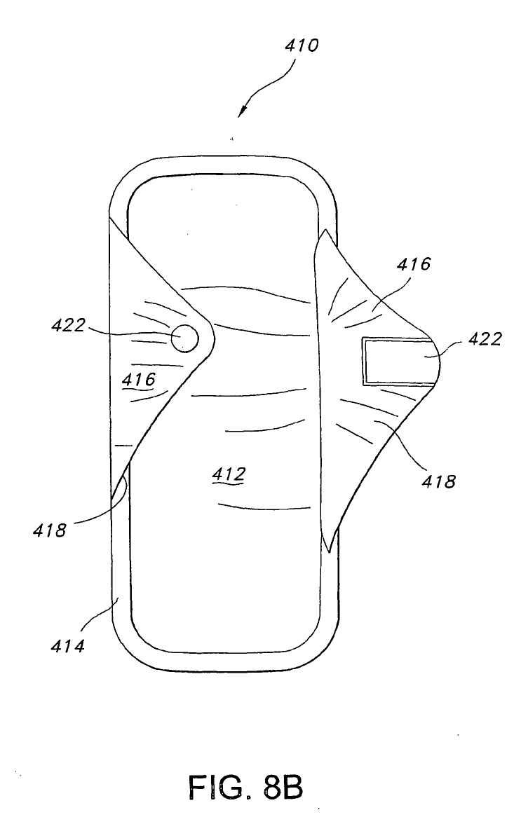

Referring now to FIG. 8, a feminine sanitary pad including substantially triangular securing tabs is disclosed. The tabs are located forward of the center of the pad to allow for greater stability and security during use. Located at the outer portion of the tabs are double-sided hook and loop type fasteners interengaging with one another so that the user may place them over the outer portion of an undergarment to hold the pad securely in place. The triangular shape of the tabs, with the widest side attached to the main portion of the device, keeps the pad stationary during physical activity and allows for longer pad length and thus greater potential absorbency.

Details of the absorbent article illustrated in FIG. 8 are provided in U.S. Provisional Application Serial No. 60/304,788 filed Jul. 13, 2001 and entitled KITE-WRAP FEMININE PAD WITH D/S HOOK AND LOOP WINGS, which is incorporated herein by reference. FIG. 8 is a top view showing the shape of the pad's fastening tabs, their location in respect to the center of the pad, and the double-sided hook and loop fasteners that hold the pad in place during use. The pad is indicated generally in FIG. 8 at 410. The main absorbent portion 412 of the pad is located on a waterproof layer 414. There are two tabs 416 laterally disposed on layer 414. The tabs are generally triangular in shape.

The bases of each of the tabs 416 attached to layer 414 are indicated at 418. These tab bases 418 are located substantially above a front to back midline, which is designated at 420. This configuration provides greater stability when the pad 410 is attached to a user's undergarment. Additionally, the relative size and shape of the tabs 416 provide for more surface area to allow the user to adjust the securement of the pad to the undergarment. Located at the ends of both the tabs 416 are the double-sided hook and loop fasteners 422. The fasteners 422 are configured such that when the user wishes to secure the pad to the undergarment, either the right tab may be placed over the left tab or the left tab may be placed over the right. The location of the fasteners 422 at the far end of the tabs 416 also allows for additional options when the user wishes to secure the pad to the undergarment.

Accordingly, the waterproof kite-shaped tabs of the embodiment illustrated in FIG. 8 are positioned toward the frontal end of the sanitary pad to help the pad remain stationary when worn. Alternatively, the positioning of the tabs may be toward the rear, in some embodiments. A central position of the loop fastener is provided on the top side with an arcuate end facing toward the outer edge. A hook button is positioned on the underside of the kite-shaped tab, directly behind the loop fastener, for ease of use when applied by a user (right to left or left to right). Accordingly, a longer sanitary pad with kite-shaped tabs that wrap around the crotch of a panty is provided, and such pad is secured with a double-sided hook and loop closure mounted on the waterproof tabs.

The forward position of the substantially triangular tabs helps to firmly secure the pad, thereby allowing for a longer length and greater absorbency. In other words, more tab surface is provided to secure the pad in the panty, especially for longer pads. The device is therefore stabilized in the correct position for heavy fluid.

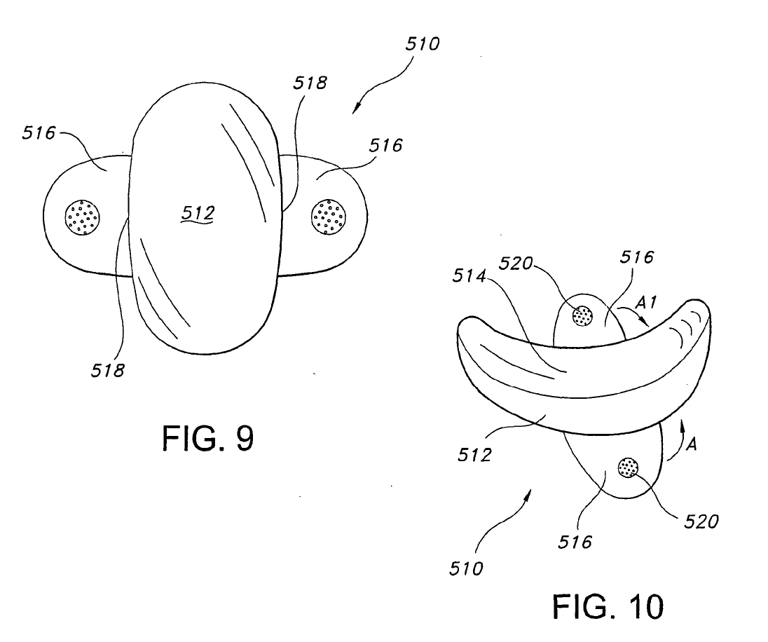

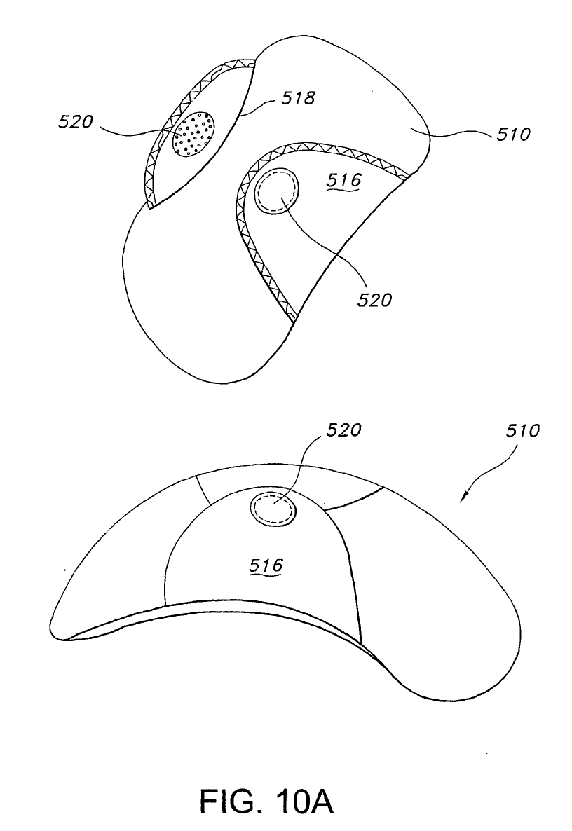

Referring now to FIGS. 9 and 10, a mini panty-liner is disclosed. The device has die-cut wraparound arcuate tabs that include double-sided hook and loop fasteners. The width of the tabs allows for greater stability when the device is used in conjunction with “fashion” underwear of the type that has a substantially thinner crotch area than “regular” underwear. The embodiment illustrated in FIGS. 9 and 10 can be configured as a reusable washable mini pad or panty-liner or as a disposable product.

Details of the embodiment illustrated in FIGS. 9 and 10 are provided in U.S. Provisional Patent Application Serial No. 60/305,895, filed Jul. 18, 2001 and entitled MINI PANTY LINER, which is incorporated herein by reference.

FIG. 9 is a bottom view of one embodiment, and FIG. 10 is a perspective view of the embodiment, which is generally indicated at 510. The liner 510 is made of a washable, reusable material that makes it attractive to environmentally conscious consumers. Alternatively, the liner 510 can be formed from material suitable for disposable use. The outer portion 512 of the liner is seen in FIG. 9 and is optionally lined with a waterproof or partially waterproof material to prevent leak-through. The inner portion 514 of the liner 510 is made of an absorbent material. It should be understood that the entire liner 510 could be absorbent and it should also be understood that a wide variety of materials could be used to manufacture the device.

A pair of arcuate tabs 516 is located laterally on the liner 510. These tabs 516 are wide at the base 518, which provides greater stability when the liner 510 is attached to the user's undergarment (not shown). The tabs are wrapped around the undergarment in the direction indicated by arrows Al in FIG. 10, leaving the inner portion 514 of the liner 510 in contact with the user's body. Located on the tabs 516 are double-sided hook and loop fasteners 520, the configuration of which allows the user to place either the right tab over the left or the left over the right. A section of loop can be die-cut proportionally larger for greater stability when used within fashion underwear. A hook fastener in the form of a coin can be provided on the underside. This embodiment of a pad product therefore has wraparound arcuate tabs with a double-sided hook and loop closure to hold the pad securely. The double-sided fastener mechanism allows the elderly or infirm to close the tab left-over-right or right-over-left.

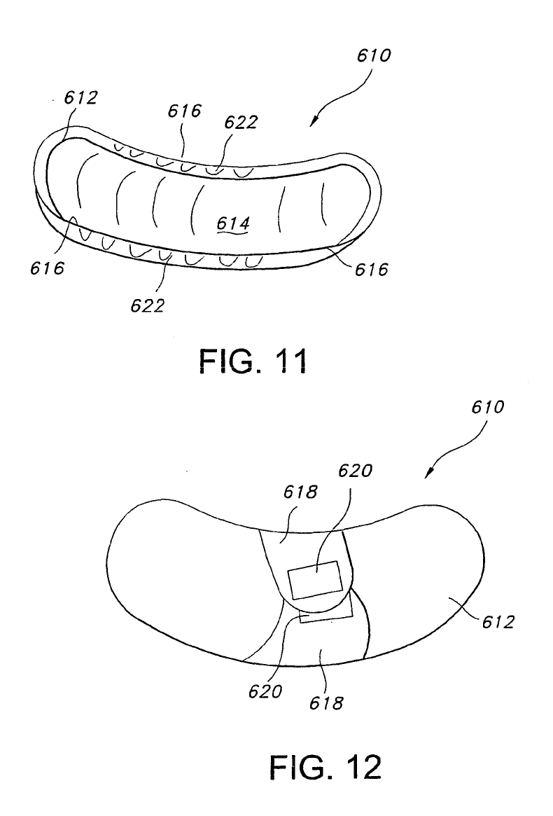

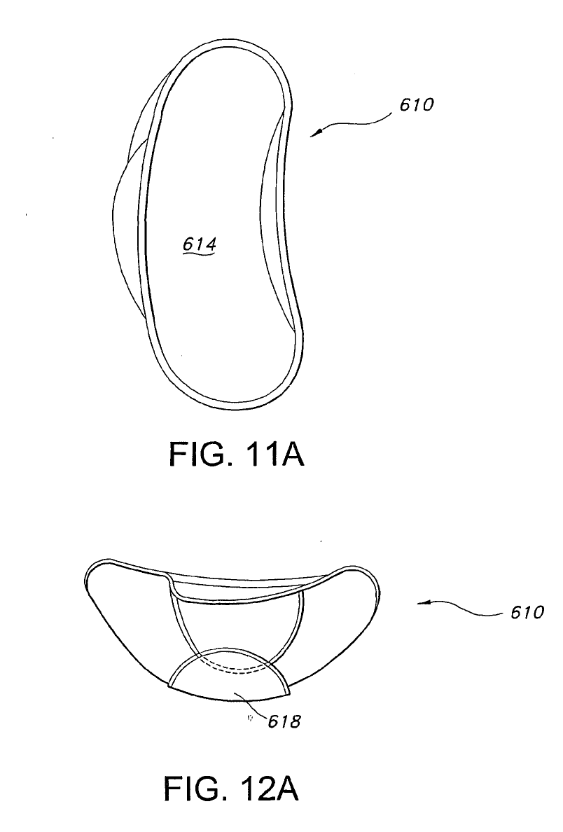

Referring now to FIGS. 11 and 12, an embodiment of an absorbent article in the form of a feminine pad is illustrated. In the illustrated embodiment, a waterproof gusset connects the various layers of the device and is further connected to tabs having double-sided hook and loop fasteners attached thereto.

Details of the embodiment illustrated in FIGS. 11 and 12 are provided in U.S. Provisional Patent Application Serial No. 60/304,791, filed Jul. 13, 2001 and entitled FEMININE PAD WITH WATERPROOF GUSSETS AND D/S HOOK AND LOOP FASTENER, which is incorporated herein by reference.

FIG. 11 is a perspective view of the absorbent product embodiment, showing the inner absorbent layers surrounded by a waterproof gusset. FIG. 12 is another perspective view of the embodiment, showing the tabs attached to the gusset and the way that they would be used to attach the device to an undergarment (not shown). The product is shown in both of FIGS. 11 and 12 and is generally indicated at 610. The waterproof gusset that attaches the two absorbent layers together is indicated in both of FIGS. 11 and 12 at 612. As shown in FIG. 11, the outer edge 616 of the gusset is elasticized to provide a smooth, comfortable fit. A pair, of absorbent layers is indicated at 614 in FIG. 11.

Turning to FIG. 12, the laterally disposed tabs 618 of the device, which are connected to the gusset 612, include double-sided hook and loop fasteners 620 that allow the user to set the pad in place on an undergarment in the manner seen in FIG. 12 (though the undergarment is not shown). The gussets extend above the sides of the absorbent layers 614 as is indicated at 622. This provides additional protection for the user. The configuration of the double-sided hook and loop fasteners shown in FIGS. 10 and 11 advantageously allows for the device to be attached to an undergarment with either one of the tabs uppermost.

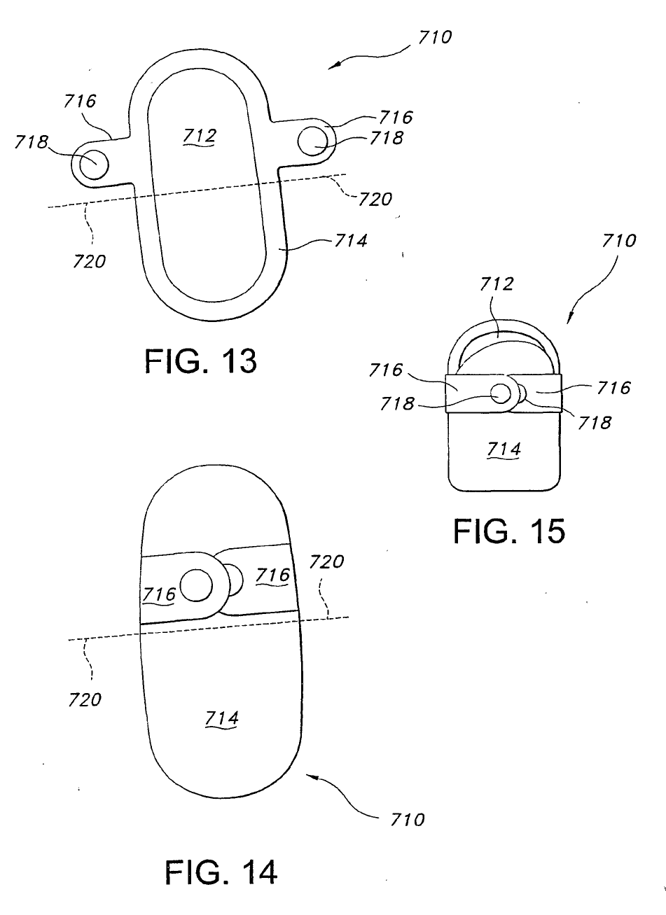

Referring now to FIGS. 13-15, a female incontinence device with double-sided hook and loop fasteners is described. The exemplary device includes a first mounted absorbing core, a waterproof outer layer to which the fasteners are attached, and a folding position for storage of the device.

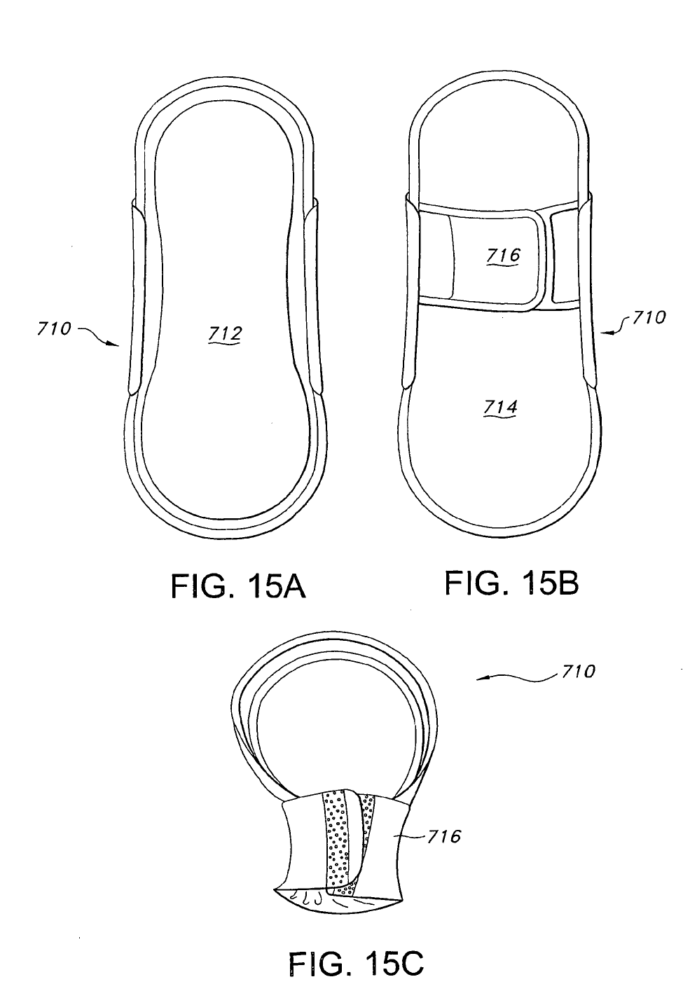

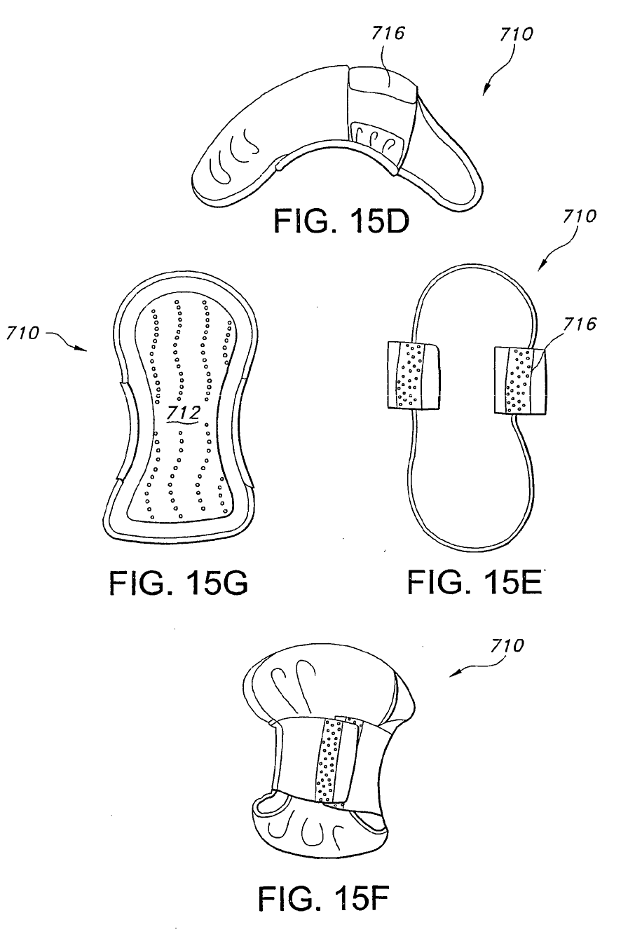

Details of the embodiment illustrated on FIGS. 13-15 are provided in U.S. Provisional Patent Application Serial No. 60/304,787, filed Jul. 13, 2001 and entitled FEMININE INCONTINENCE PAD WITH D/S HOOK AND LOOP AND STORAGE, which is incorporated herein by reference. FIG. 13 illustrates the absorbent layer, the outer waterproof layer, and the double-sided hook and loop fasteners of an exemplary embodiment of the device. FIG. 14 is a view showing the outer waterproof layer with the hook and loop fasteners attached to one another in the same manner that they would be used to attach the device to an undergarment (not shown). FIG. 15 is a view of the exemplary embodiment in the folded position.

Referring to FIG. 13, the absorbent article embodiment is generally indicated at 710. The absorbent core is indicated at 712, with the outer waterproof layer at 714. Attached to the outer, waterproof layer are the lateral tabs 716 that include double sided hook and loop fasteners 718. A front-back midline demarcation is indicated at 720, and the lateral tabs 716 are positioned substantially on one side thereof. This is also seen in FIG. 14.

The configuration of the double sided hook and loop fasteners allows the user to attach the device to an undergarment (not shown) in the manner seen in FIG. 14 with either of the tabs overlying the other, i.e., right over the left or vice versa. The placement of the tabs 716 on one side of the midline demarcation 720 allows for the device to be folded for storage, shipping, or the like, as is seen in FIG. 15. The absorbent article 710 is contemplated to be dimensioned such that it may be worn with “thong” underwear, which has a substantially narrower crotch area than standard underwear.

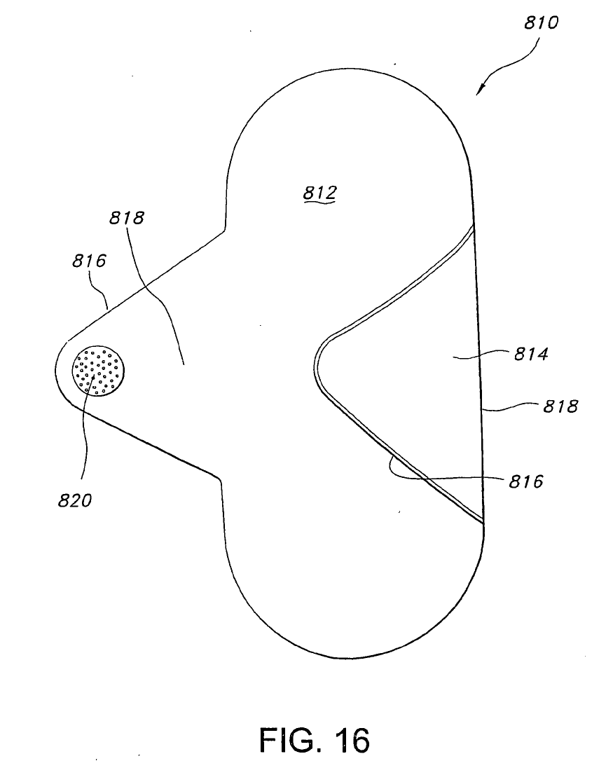

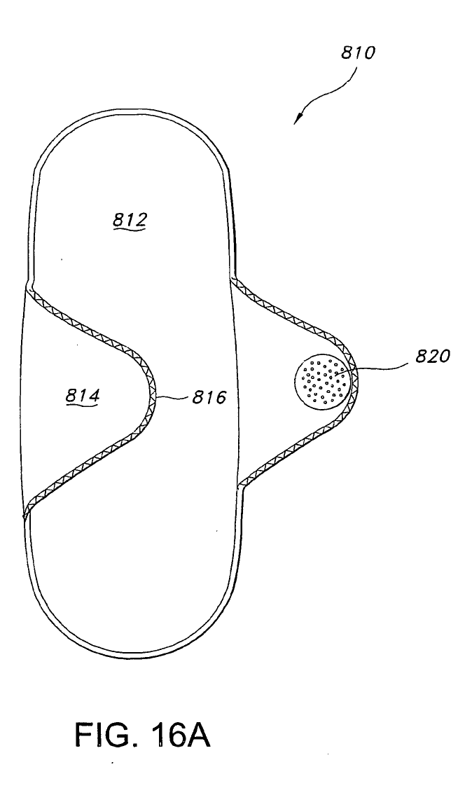

Referring now to FIG. 16, another embodiment of a panty-liner with broad lateral attachment tabs is disclosed. The embodiment illustrated in FIG. 16 includes coin-shaped hook portions that interact with the soft “loop” side of the device for attaching the liner to an undergarment. This embodiment is particularly advantageous as a feminine care product, specifically for menstruating or incontinent women. This embodiment can be configured as a reusable, washable liner or a disposable liner with a waterproof side and an absorbent side that includes broad laterally disposed tabs for attaching the device to the user's undergarment.

Details of the embodiment illustrated in FIG. 16 are provided in U.S. Provisional Patent Application Serial No. 60/305,847, filed Jul. 18, 2001 and entitled PANTY-LINER, which is incorporated herein by reference.

FIG. 16 is a top view of an embodiment with one of the tabs folded over to show the absorbent side. The coin-shaped “hook” attachment means is seen on the other tab. In FIG. 16, the embodiment of the absorbent article is generally indicated at 810. The novel panty liner has a waterproof side 812 and an absorbent side 814. The article can be made of a washable material so that it may be used over instead of being thrown away. Alternatively, it may be configured for single use applications. With respect to the shape of the laterally disposed tabs 816, the tab base 818 is wide to allow for greater stability when the device is attached to an undergarment. Located on the waterproof side of the liner 810, there is a coin-shaped hook portion 820 that makes up part of the hook and loop fastening or engagement means. The absorbent side 814 is made of a material that provides the cooperating “loop” area when the hook fastener 820 is brought over the outside of the undergarment (not shown). A guard or shield product with wraparound tabs, and that has double-sided hook and loop closure to hold the guard securely, is therefore provided according to this embodiment of the invention.

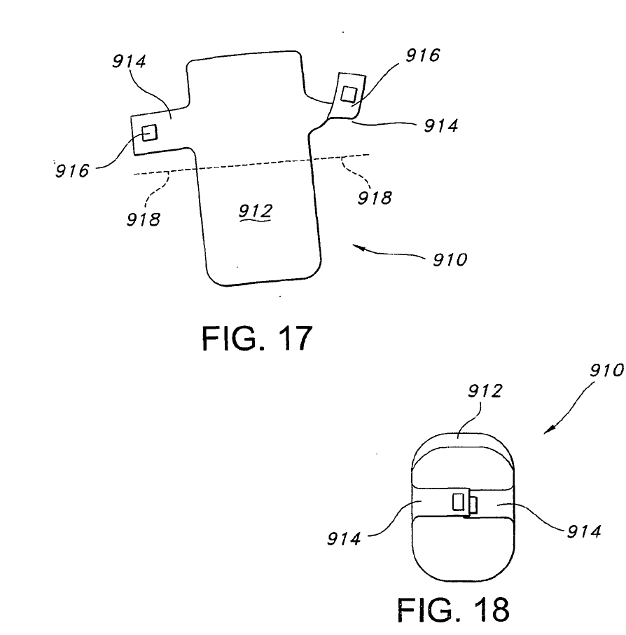

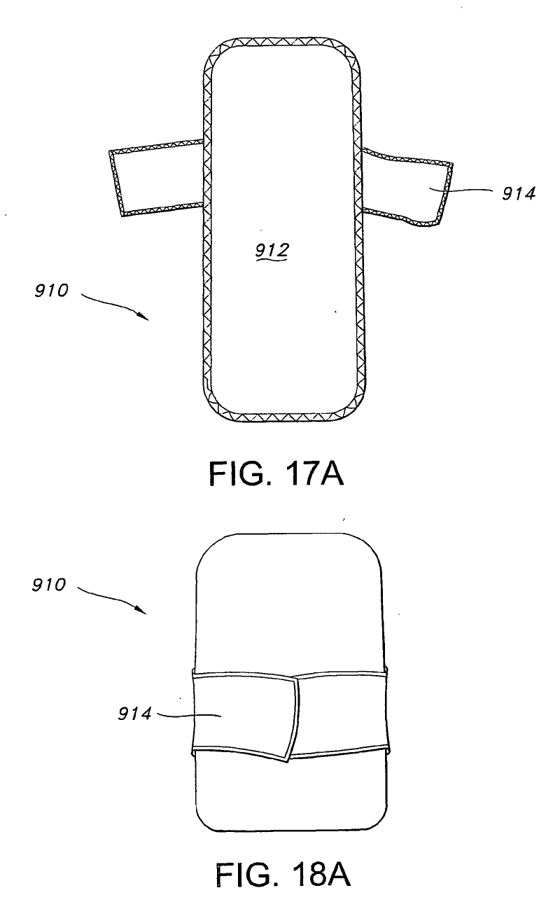

Referring to FIGS. 17 and 18, an embodiment of a feminine pad with tabs is disclosed. The tabs carry single- or double-sided hook and loop fasteners and the tabs are additionally located on one side of the front to back midline, so that the device may be optionally folded for storage or shipping. The illustrated embodiment can be configured for use during menstruation. More specifically, this embodiment provides a slim line feminine pad that has a folding feature related to the location of the attachment tabs on the main body of the device.

Details of the embodiment illustrated in FIGS. 17 and 18 are provided in U.S. Provisional Patent Application Serial No. 06/304,770, filed on Jul. 13, 2001 and entitled FEMININE SLIM LINE MAXI-PAD WITH SOFT-CROSS TABS FEATURES FOLDED POSITION FOR STORAGE, which is incorporated herein by reference.

FIG. 17 is a view of the embodiment showing the laterally disposed tabs on the main body portion of the device. FIG. 18 is a view of the embodiment in the folded or storage configuration. Referring to FIGS. 17 and 18, the embodiment of the absorbent article is generally indicated at 910. The main body of the device, the part that includes an absorbent portion, is designated at 912. Located laterally of the main body 912 are the attachment or fastening tabs 914. These tabs carry on them double-sided hook and loop fasteners 916. The construction of the fasteners 916, with the hook portion of the fastener on one side and the loop portion located on the opposite side, allows the user to fasten the device 910 to an undergarment (not shown) with either one of the tabs uppermost. The lateral tabs 914 are located substantially on one side of the midline 918. This allows for the device 910 to be folded, either for storage, for ease in carrying, or for shipping. The folded device 910 is shown in FIG. 18.

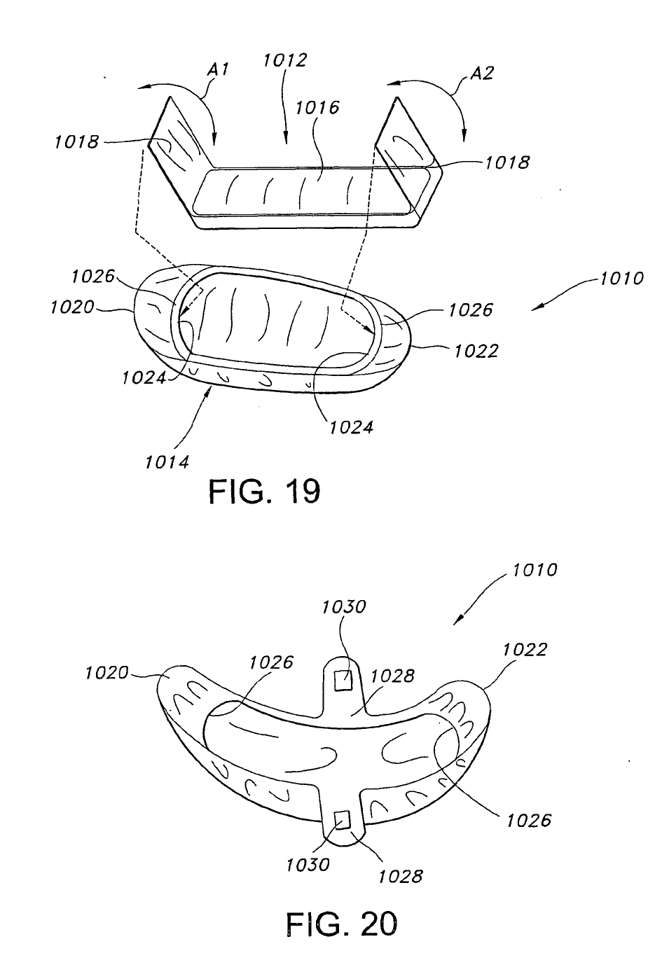

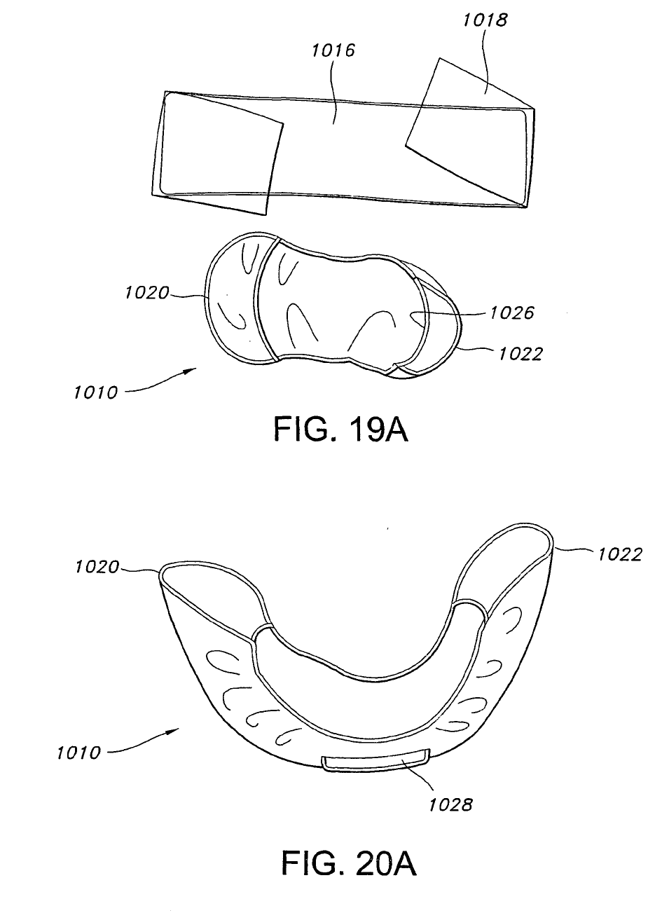

Referring to FIGS. 19 and 20, an embodiment of a dual system for a panty-liner and pouch is disclosed. In this embodiment, the panty-liner is separate from the waterproof pouch and includes cuffs that fit into the pouch. The waterproof pouch is also connected to the tabs that carry the double-sided hook and loop fasteners that allow the entire assembled unit to be attached to the user's undergarment. The illustrated embodiment is therefore beneficially used in feminine products such as panty-liners. More specifically, it may be configured to provide a panty liner and pouch system where the liner is removable from the waterproof pouch. Additionally, the pouch is connected to laterally disposed tabs that allow the unit to be attached to the user's undergarments.

Details of the embodiment shown in FIGS. 19 and 20 are provided in U.S. Provisional Patent Application Serial No. 06/304,769; filed Jul. 13, 2001 and entitled DUEL SYSTEM:PANTY-LINER AND POUCH, which is incorporated herein by reference. FIG. 19 is an exploded perspective view of the embodiment of the panty-liner and pouch system with the two elements separated from one another. FIG. 20 is a perspective view of the panty-liner and pouch system with the two elements together and showing the laterally disposed tabs with the double-sided hook and loop fasteners.

In FIGS. 19 and 20, the panty-liner and pouch system is generally indicated at 1010. In FIG. 19, the liner portion is indicated at 1012, and the pouch is indicated at 1014. The liner includes a main absorbent portion 1016 and two cuff areas on either end, indicated at 1018. These two cuff portions 1018 are foldable, as indicated by directional arrows A1 and A2. The pouch portion 1014 is waterproof and has a front end 1020 and a rear end 1022. Located at both the front and rear ends 1020, 1022 are the cuff receiving pockets 1024. These cuff receiving pockets 1024 are elasticized on their edges 1026 to hold the cuffs 1018 firmly in place when the liner portion 1012 is placed in the pouch 1014.

Directional arrows A3 and A4 show how the cuffs 1018 are placed in the cuff receiving pockets 1024. Located on the lateral sides of the pouch 1014 are the tabs 1028, seen in FIG. 20. Located on the tabs 1028 are double-sided hook and loop fasteners 1030 that allow the user to attach the panty-liner and pouch system 1010 to an undergarment (not shown). The double sided configuration allows either one or the other of the tabs 1028 to be placed over its partner.

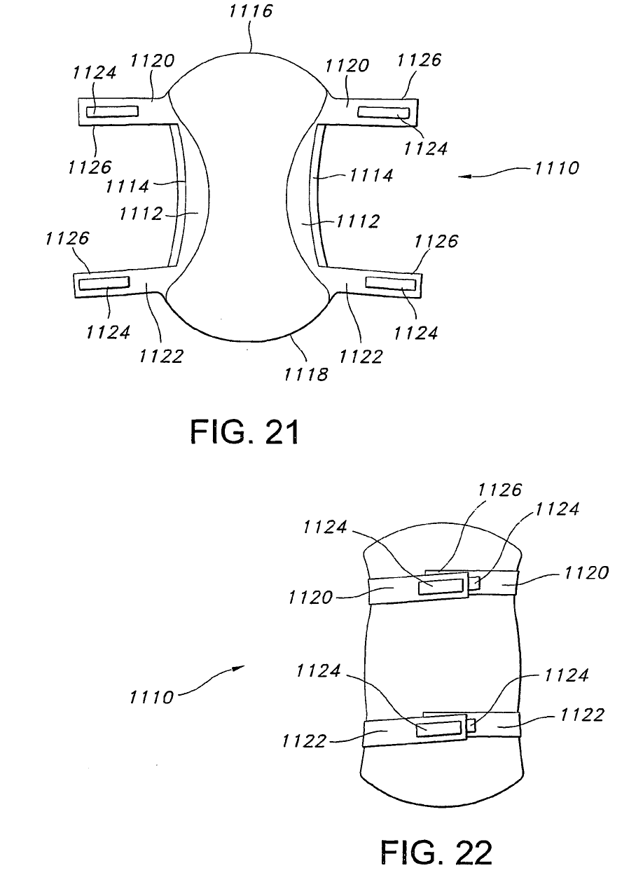

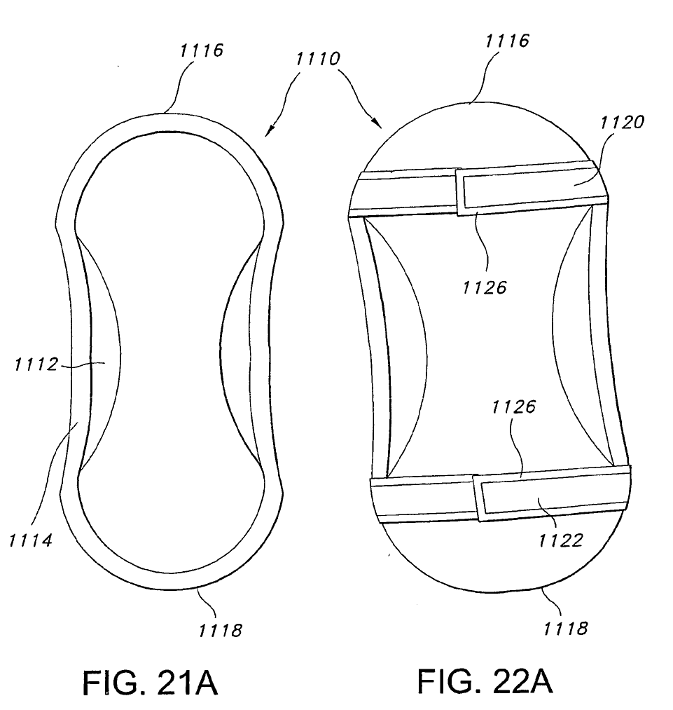

Referring now to FIGS. 21 and 22, an embodiment of a gusseted panty-liner is disclosed. Front and back straps secure the device in the desired position. Double-sided hook and loop fasteners are used to attach the panty-liner in place. The device also includes a side gusset of waterproof, breathable material.

Details of the embodiment illustrated in FIGS. 21 and 22 are provided in U.S. Provisional Patent Application Serial No. 60/305,877 filed Jul. 18, 2001 and entitled GUSSETED PANTY-LINER, which is incorporated herein by reference. FIG. 21 is a top view of the panty-liner embodiment, and FIG. 22 is a bottom view of the same embodiment showing its straps attached to one another. Turning to FIG. 21, the embodiment is configured for use as a gusseted panty liner and is indicated at 1110. In FIG. 21, on each side of the absorbent portion of the device is the gusset portion 1112. This is made from breathable and waterproof material and includes an elastic edge 1114 to provide a more secure fit. The liner 1110 has a front 1116 and a back 1118. Located proximate the front 1116 are the front straps 1120 and located proximate the back 1118 are the back straps 1122. Both the straps 1120 and 1122 optionally have double-sided hook and loop fasteners that are indicated at 1124. This optional use of double-sided fasteners allows the user to put either the left strap or tab over the right strap or vice versa.

The outer edges of the straps optionally have a soft area 1126, which may extend to include the entire strap, for comfort. Comfort is also provided by side gussets formed of breathable and waterproof material and having an elasticized outer edge. The fasteners of this embodiment are oriented in parallel positions using a double sided hook and loop mechanism. More specifically, the following construction is employed in the illustrated exemplary embodiment: loop on top, hook on underside, and soft material over the locked edges of the fasteners for a soft touch.

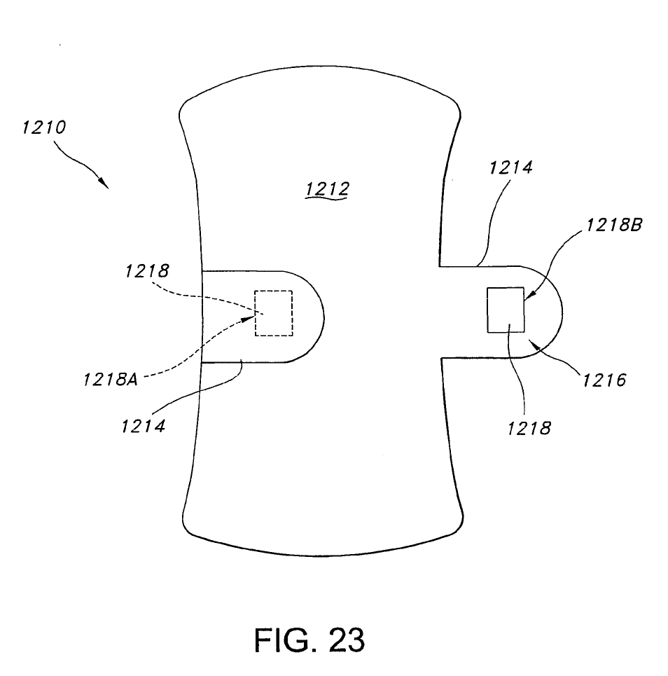

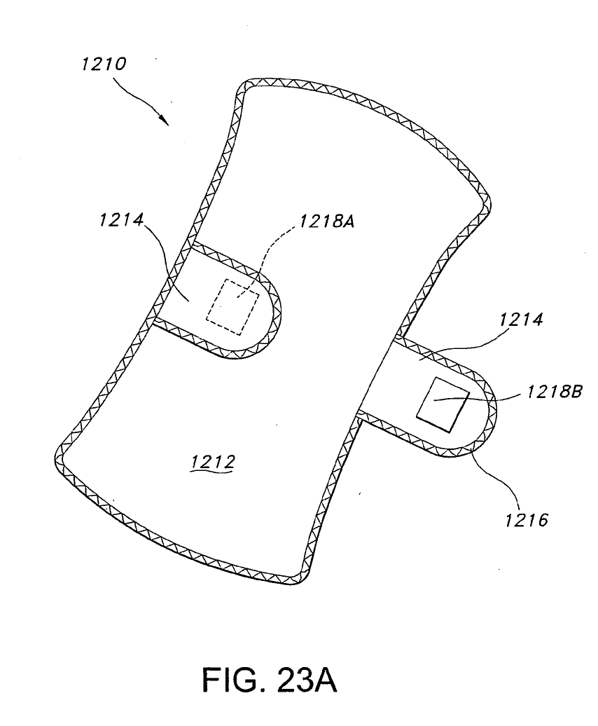

Referring to FIG. 23, yet another embodiment will be described. More specifically, the illustrated embodiment is configured as a mini-pad used during a woman's menstrual period. More specifically, the embodiment provides a mini-pad that includes die-cut tabs having double-sided hook and loop fasteners disposed thereon. The double-sided configuration allows for either right tab over left or left tab over right engagement.

The embodiment illustrated in FIG. 23 is described in U.S. Provisional Patent Application Serial No. 60/304,789, filed Jul. 13, 2001 and entitled REUSUABLE MINI-PAD, which is incorporated herein by reference. FIG. 23 is a top view showing one of the die-cut tabs of the illustrated embodiment extended and the other of the die-cut tabs turned over so that the interengaging hook and loop type fasteners are clearly seen. The embodiment is indicated in FIG. 23 at 1210.

A main body portion 1212 of the absorbent article includes two die-cut or otherwise formed tabs 1214 that are laterally disposed. It is contemplated that the outer edge of these tabs 1214 would have overlocked thread or other soft edges as is indicated at 1216. Interengaging hook and loop type fasteners 1218 are located on the tabs 1214. In the embodiment shown in FIG. 23, on the left side of the Figure are the laminated loops 1218A and on the right side of FIG. 23 are the hooks 1218B. The disposition of the hooks and loops allows for the user to put either the left tab over the right or the right tab over the left, depending on the convenience and/or the user's handedness.

The loop is optionally laminated on the article's underside. The hook is provided on the underside of die-cut loop portion. Overlocked thread or other soft edges are provided on the die-cut tab. The double sided hook and loop fastening system of this embodiment may be advantageous because it allows right-over-left or left-over-right fastening.

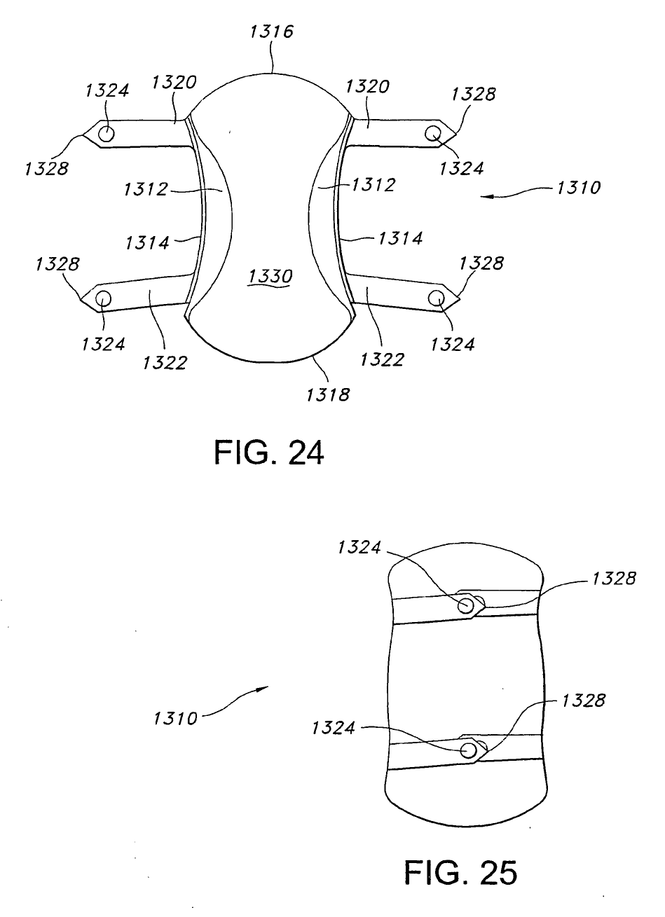

Referring to FIGS. 24 and 25, an embodiment of a gusseted panty-liner is disclosed. Front and back straps of the liner secure the device in the desired position. The straps of the illustrated embodiment have pointed triangular distal ends. Double-sided hook and loop fasteners are located near these triangular ends and are used to attach the panty-liner in place. The device also includes a side gusset of waterproof, breathable material. The embodiment illustrated in FIGS. 24 and 25 relates to feminine hygiene products, specifically to panty liners with front and back straps to secure the device in place, but the features of the illustrated embodiment also apply to other types of absorbent articles. The front and back straps have pyramidal or triangular ends with double-sided hook and loop fasteners located near those ends.

Details of the embodiment illustrated in FIGS. 24 and 25 are provided in U.S. Provisional Patent Application Serial No. 60/305,848, filed Jul. 18, 2001 and entitled INCONTINENCE PAD WITH WRAP-AROUND D/S HOOK AND LOOP CLOSURE, which is incorporated herein by reference. FIG. 24 is a top view of the subject embodiment, and FIG. 25 is a bottom view showing the straps or tabs attached to one another. Turning to FIG. 24, the illustrated embodiment is a gusseted panty liner indicated at 1310. In FIG. 24, on each side of the absorbent portion of the device is a gusset portion 1312. This is optionally made from breathable and waterproof material and includes an elastic edge 1314 to provide a more secure fit. This area channels fluid to the absorbent area of the pad 1330.

The liner 1310 has a front 1316 and a back 1318. Located proximate the front 1316 are the front straps 1320 and located proximate the back 1318 are the back straps 1322. The straps 1320 and 1322 both have generally triangular shaped ends 1328. Both the straps 1320 and 1322 optionally have single- or double-sided hook and loop fasteners that are indicated at 1324. In the embodiment illustrated in FIGS. 24 and 25, the hook and lop fasteners are rounded or circular. In addition the are double sided, allowing the user to put either the left strap or tab over the right strap or vice versa.

Accordingly, the embodiment illustrated in FIGS. 24 and 25 provides a guard or shield product with wraparound tabs that has a single- or double-sided hook and is loop closure to hold the guard securely. The illustrated elasticized waterproof gussets help to channel fluid as described previously.

Although the invention is illustrated and described herein with reference to specific embodiments, the invention is not intended to be limited to the details shown. Rather, various modifications may be made in the details within the scope and range of equivalents of the claims and without departing from the invention. For example, although certain embodiments disclosed herein are configured for reusable applications, the features of those embodiments apply equally to disposable products. Likewise, although certain embodiments disclosed herein are configured for disposable applications, the features of those embodiments apply equally to reusable products.

What is claimed is:

1. An absorbent article comprising:
a body portion having a perimeter and a garment-facing surface; and
a pair of fastening tabs extending from said garment-facing surface of said body portion at locations spaced inwardly from said perimeter of said body portion, each of said fastening tabs having means for engaging the other of said fastening tabs, and said fastening tabs being positioned to receive a portion of a garment therebetween.

2. The absorbent article of claim 1, said engaging means comprising an adhesive, hooks, or a snap.

3. The absorbent article of claim 1, said fastening tabs each comprising opposed surfaces, one of said surfaces carrying hooks and the other of said surfaces being engageable by hooks.

4. The absorbent article of claim 1, said fastening tabs being end portions of a component of said absorbent article.

5. The absorbent article of claim 1, said fastening tabs being separate from one another.

6. The absorbent article of claim 1, said fastening tabs being coupled to, or integral with, a layer of said body portion.

7. The absorbent article of claim 1, said body portion having a width, and the space between said fastening tabs being smaller than the width of said body portion.

8. The absorbent article of claim 7, said space between said fastening tabs being selected to receive a portion of a thong-style garment.

9. An absorbent article comprising:
a body portion having a perimeter and a garment-facing surface; and
fastening tabs extending from said garment-facing surface of said body portion at locations spaced inwardly from said perimeter of said body portion, said fastening tabs each having opposite surfaces, one of said surfaces carrying hooks and the other of said surfaces being engageable by hooks, and said fastening tabs being positioned to receive a portion of a garment therebetween.

10. The absorbent article of claim 9, said fastening tabs being end portions of a component of said absorbent article.

11. The absorbent article of claim 9, said fastening tabs being separate from one another.

12. The absorbent article of claim 9, said fastening tabs being coupled to a layer of said body portion.

13. The absorbent article of claim 9, said body portion having a width, and the space between said fastening tabs being smaller than the width of said body portion.

14. The absorbent article of claim 13, said space between said fastening tabs being selected to receive a portion of a thong-style garment.

15. The absorbent article of claim 1, said engaging means being positioned on said fastening tabs to allow engagement between said fastening tabs independent of which fastening tab is positioned on top of the other.

16. The absorbent article of claim 9, said hooks being positioned to allow engagement between said tabs independent of which tab is positioned on top of the other.

17. A fastening tab for an absorbent article, said fastening tab comprising opposite surfaces, one of said surfaces carrying hooks and the other of said surfaces being engageable by hooks, wherein said hooks are spaced from at least one edge portion of said fastening tab.

18. The fastening tab of claim 17 comprising an elastic material.

19. The fastening tab of claim 17 comprising a woven or non-woven substrate layer from which said hooks extend, wherein said substrate is engageable by said hooks.

20. The fastening tab of claim 17, said hooks being positioned in a region to define a border between an edge portion of said fastening tab and said hooks, said border being devoid of hooks and said border substantially surrounding said region.

21. The fastening tab of claim 20, said region being substantially circular.

22. The fastening tab of claim 20, said region being positioned at an end portion of said fastening tab.

23. The fastening tab of claim 22, said edge portion of said fastening tabs having an arcuate shape.

24. The absorbent article of claim 1, said absorbent article being selected from the group consisting of adult diapers, baby diapers, adult absorbent underwear, children's absorbent underwear, panty liners, panty shields, maxi pads, mini pads, and incontinence garments.
